(19) United States
(12) Patent Application Publication
(10) Pub. No.: US 2023/0374115 A1
(43) Pub. Date: Nov. 23, 2023

(54) NOVEL CORONAVIRUS RBD SPECIFIC MONOCLONAL ANTIBODY AND LINEAR EPITOPE AND APPLICATION THEREOF

(71) Applicants: Yulin Feng, Beijing (CN); CHONGQING MEDICAL UNIVERSITY, Chongqing (CN)

(72) Inventors: Aishun Jin, Chongqing (CN); Tingting Li, Chongqing (CN); Xiaojian Han, Chongqing (CN); Yingming Wang, Chongqing (CN); Chao Hu, Chongqing (CN); Shenglong Li, Chongqing (CN); Jianwei Wang, Chongqing (CN)

(21) Appl. No.: 18/111,434

(22) Filed: Feb. 17, 2023

Related U.S. Application Data
(63) Continuation of application No. PCT/CN2020/115480, filed on Sep. 16, 2020.

(30) Foreign Application Priority Data
Aug. 19, 2020 (CN) 202010839226.6
Aug. 19, 2020 (CN) 202010839857.8

(51) Int. Cl. C07K 16/10

(52) U.S. Cl. CPC C07K 16/1002; C07K 2317/34

(57) ABSTRACT

The present disclosure belongs to the field of immune technology, and particularly discloses a SARS-CoV-2 RBD-specific monoclonal antibody including a heavy chain having an amino acid sequence set forth in SEQ ID NO: 1 and a light chain having an amino acid sequence set forth in SEQ ID NO: 2. The present disclosure further discloses a linear epitope of the SARS-CoV-2 RBD-specific monoclonal antibody having an amino acid sequence set forth in SEQ ID NO: 3. The SARS-CoV-2 RBD-specific monoclonal antibody and the linear epitope thereof of the present disclosure are critical for research and development of vaccines and micromolecule drugs, diagnosis, prevention and treatment of COVID-19.

### TECHNICAL FIELD

The present disclosure belongs to the field of immune technology, and particularly relates to a SARS-CoV-2 RBD-specific monoclonal antibody, a linear epitope thereof and use thereof.

### BACKGROUND

SARS-CoV-2 is a coronavirus of Coronaviridae, Nidovirales. It has the largest genome of a length of 27 to 32 kb among known RNA viruses, and consists of at least 4 major structural proteins including spike protein (S protein), membrane protein (M protein), envelope protein (E protein) and nucleocapsid protein (N protein). The virus enters cells depending on the S protein and the receptor binding domain (RBD) of the S protein. The S protein comprises two subunits 51 and S2. The receptor binding domain (RBD) is located on the subunit 51 and it is required for the recognition of the host cell surface receptors and mediation of the fusion with the host cells. The N protein is a basic phosphoprotein, of which the central region binds to the viral genomic RNA to form a nucleocapsid helix, and is the core structure wrapping the viral genetic material and one of the viral proteins with the highest expression in infected cells.

At present, no specific medicine for the pathogen of COVID-19 is available, and the research and development of vaccines are still underway. Recently, specific neutralizing antibodies of high concentration were found in the plasma of patients in convalescence. When administered to other patients, the plasma could neutralize the pathogen and mediate effective immune response. The proper use of the plasma of patients in convalescence is thus expected to provide an effective treatment for patients infected with SARS-CoV-2, reduce the mortality and save lives.

Chinese Patent Publication No. CN111303280A discloses a full human monoclonal antibody against SARS-CoV-2 with high neutralizing activity, which recognizes a non-RBD region in S1. Since SARS-CoV-2 enters host cells through the binding of RBD to ACE2 of the host cells, the full human monoclonal antibody of the above patent has limited blocking effect on the virus. In addition, the above patent acquires the antibody cDNA by labeling plasmocytes. Compared to plasmocytes, memory B cells respond faster after activation and can induce a humoral immune response that is faster and stronger, while plasmocytes only induce limited humoral immune response.

Moreover, the research on the epitopes of SARS-CoV-2 is of great importance to the prevention, detection, diagnosis and treatment of COVID-19. Chinese Patent Publication No. CN111440229A discloses a T cell epitope of SARS-CoV-2. In that patent, T cell epitopes of SARS-CoV-2 N protein were predicted by Class I Immunogenity tool of IEDB Analysis Resource, and 600 immunogenic and 181 non-immunogenic 9-mer peptides were analyzed. However, no linear epitope recognized by the B cells has been reported at present.

### SUMMARY

An object of the present disclosure is to provide a SARS-CoV-2 RBD-specific monoclonal antibody that can be recognized by B cells, a linear epitope thereof and use thereof.

For the above object, technical schemes of the present disclosure are as follows:

For the above object, the present disclosure provides a SARS-CoV-2 RBD-specific monoclonal antibody comprising a heavy chain having an amino acid sequence set forth in SEQ ID NO: 1 and a light chain having an amino acid sequence set forth in SEQ ID NO: 2 (mAb 3-CQTS126).

Preferably, the SARS-CoV-2 RBD-specific monoclonal antibody is obtained by sorting RBD-specific memory B cells and acquiring antibody variable region cDNA from mRNA of the RBD-specific memory B cells.

The present disclosure further provides use of the SARS-CoV-2 RBD-specific monoclonal antibody in preparing a reagent, a vaccine or a medicament for detecting or diagnosing SARS-CoV-2. The medicament includes the SARS-CoV-2 RBD-specific monoclonal antibody and a pharmaceutically acceptable excipient, diluent or carrier. The present disclosure further provides a nucleic acid molecule encoding the SARS-CoV-2 RBD-specific monoclonal antibody. The present disclosure further provides an expression cassette, a recombinant vector, a recombinant bacterium or a transgenic cell line comprising the nucleic acid molecule. The present disclosure further provides use of the expression cassette, the recombinant vector, the recombinant bacterium or the transgenic cell line in preparing a product.

The present disclosure further provides a product comprising the SARS-CoV-2 RBD-specific monoclonal antibody. The product is used for any of the following (b1)-(b4): (b1) binding to SARS-CoV-2; (b2) detecting SARS-CoV-2; (b3) binding to SARS-CoV-2 S protein; and (b4) detecting SARS-CoV-2 S protein.

The present disclosure further discloses a linear epitope of the SARS-CoV-2 RBD-specific monoclonal antibody having an amino acid sequence set forth in SEQ ID NO: 3.

Preferably, the linear epitope is obtained by following steps: denaturing the SARS-CoV-2 S protein or SARS-CoV-2 RBD protein; binding the SARS-CoV-2 RBD-specific monoclonal antibody to the SARS-CoV-2 S protein or the SARS-CoV-2 RBD protein subjected to denaturation; and performing antigenic linear epitope section synthesizing of the S protein or the RBD protein to obtain the linear epitope.

The present disclosure further provides a nucleic acid encoding the linear epitope and a recombinant vector comprising the nucleic acid.

Principle and the beneficial effects of the present disclosure are as follows:
(1) Compared with monoclonal antibodies targeting non-RBD regions in 51, the monoclonal antibody of the present disclosure binds to RBD, and provides a broader application range of antibody drug screening, diagnosis, prevention and treatment for COVID-19.
(2) The monoclonal antibody provided by the present invention is obtained by sorting RBD-specific memory B cells, and compared with methods of sorting plasmocytes to give monoclonal antibodies in the prior art, the monoclonal antibody prepared by the present invention can induce stronger humoral immune response. In addition, in the present invention, subsequent RT-PCR, nested PCR and antibody function analysis are only performed on RBD-specific memory B cells, thus greatly improving the specific binding capacity of the monoclonal antibody to RBD.
(3) The linear epitope obtained according to the present disclosure is prospective in a broader application range of detection, diagnosis, and vaccine and therapeutic research and development for SARS-CoV-2. For example, the linear epitope according to the present disclosure can be used for measuring antibody titer in a patient and detecting a humoral immune response state after SARS-CoV-2 vaccination, and can be used for research and development of micromolecule drugs and vaccines.

### BRIEF DESCRIPTION OF THE DRAWINGS

FIG. 1 shows a cell sorting diagram of analyzing RBD-specific memory B cells by a flow cytometry;

FIG. 2 shows a cell sorting diagram of analyzing RBD-specific memory B cells by a flow cytometry;

FIG. 3 shows an electropherogram of single-cell antibody gene PCR products, where 1-24, 25-48 and 49-72 represent well numbers of electrophoresis;

FIG. 4 shows a agarose gel electropherogram of an antibody gene expression cassette containing CMV promoters, WPRE-gamma or WPRE-kappa elements after PCR amplification;

FIG. 5 shows RBD-specific assay results of CQTS126;

FIG. 6 shows ELISA results for binding of the SARS-CoV-2 RBD-specific monoclonal antibody to SEQ ID NO: 3;

FIG. 7 shows results of Experiment I in which SEQ ID NO: 3 is bound in the plasma of patients but not in that of healthy subjects;

FIG. 8 shows results of Experiment II in which SEQ ID NO: 3 is bound to a RBD receptor ACE2.

### DETAILED DESCRIPTION

The present disclosure is further detailed by way of specific embodiments:

### Example 1

This example provides a SARS-CoV-2 RBD-specific monoclonal antibody (mAb 1-CQTS126) comprising a heavy chain amino acid sequence set forth in SEQ ID NO: 1 and a light chain amino acid sequence set forth in SEQ ID NO: 2.

This example further provides use of the SARS-CoV-2 RBD-specific monoclonal antibody in preparing a reagent or a medicament for detecting or diagnosing SARS-CoV-2.

In practical manufacturing, the SARS-CoV-2 RBD-specific monoclonal antibody obtained in this example can be used to prepare a nucleic acid molecule, prepare an expression cassette, a recombinant vector, a recombinant bacterium or a transgenic cell line comprising the nucleic acid molecule, or prepare a pharmaceutical composition comprising the SARS-CoV-2 RBD-specific monoclonal antibody and a pharmaceutically acceptable excipient, diluent or carrier.

In application, the SARS-CoV-2 RBD-specific monoclonal antibody obtained in this example can be used to prepare a product that may possess use for any of the following (b1)-(b4): (b1) binding to SARS-CoV-2; (b2) detecting SARS-CoV-2; (b3) binding to a SARS-CoV-2 S protein; and (b4) detecting the SARS-CoV-2 S protein.

This example further provides a method for screening the SARS-CoV-2 RBD-specific monoclonal antibody, including: sorting a single RBD-specific memory B cell from peripheral blood of a patient who has recovered from COVID-19, acquiring mRNA of the RBD-specific memory B cell, constructing an antibody variable region gene expression cassette through RT-PCR and nested PCR, transferring the antibody variable region gene expression cassette into 293T cells to express an antibody, collecting supernatant, detecting RBD specificity of the supernatant by ELISA, and screening to obtain the RBD-specific monoclonal antibody. Specifically, the method includes following steps:

S1, peripheral blood samples of several patients who had recovered from COVID-19 were collected; PBMCs were separated and frozen in a freezer at −80° C. for later use.

S2, Dead cells of the PBMCs obtained in the S1 were removed by using a dead dye; live memory B cells with high specificity and binding capacity to RBD in the PBMCs were stained and labeled with CD19, mIg-G, mIg-D and RBD to screen out the RBD-specific memory B cells; the specific memory B cells were sorted by a flow cytometry onto 96-well plates at one specific memory B cell per well, and frozen in a freezer at −80° C. for later use.

Specifically, a concentration range of the dead dye of this example in staining is preferably 1-2 μg/mL, more preferably 1.5 μg/mL; CD19 is a B cell marker supplied by Biolegend, and a concentration range in staining is 1-2 μg/mL, preferably 1.5 μg/mL in this example. mIg-G is a B cell surface receptor supplied by Biolegend, and the concentration range in staining is 1-2 μg/mL, preferably 1.5 μg/mL in this example; mIg-D is a B cell surface receptor supplied by Biolegend, and a concentration range in staining is 1-2 μg/mL, preferably 1.5 μg/mL in this example; RBD is a SARS-CoV-2 receptor-binding domain supplied by Sino Biological, and a concentration range in staining is 1-2 μg/mL, preferably 1.5 μg/mL in this example.

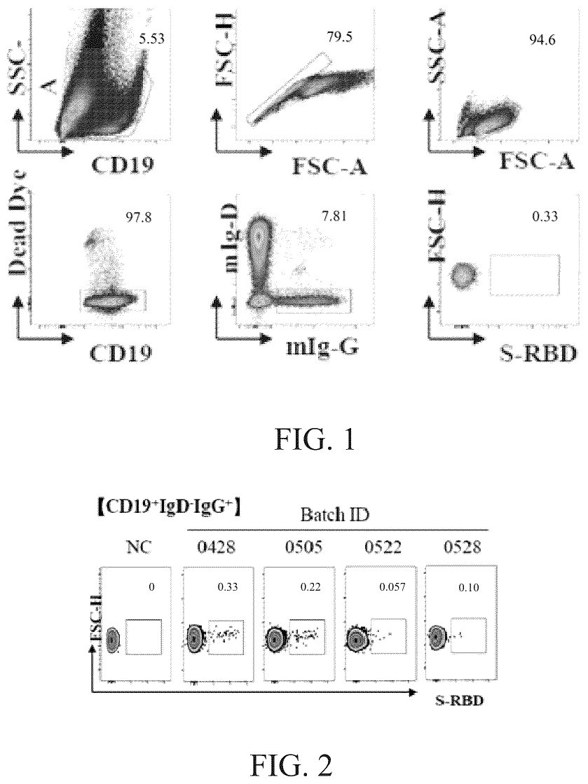

The RBD-specific memory B cells were sorted by the flow cytometry. Cells in the PBMCs were sorted using CD19, mIg-G, mIg-D and S-RBD to obtain memory B cells specific for RBD, as shown in FIGS. 1 and 2. Batch IDs 0428, 0505, 0522 and 0528 in FIG. 2 were selected batches. The rationale of screening RBD-specific memory B cells using CD19, mIg-G, mIg-D and S-RBD in this example is as follows: the PBMCs were stained with the dead dye to remove dead cells, and live memory B cells expressing RBD-specific IgG in the PBMCs were stained and labled with a B cell marker CD19 and a memory B cell markers mIg-G (positive) and a mIg-D (negative). CD19 cell population was separated by the flow cytometry; mIg-G⁺mIg-D⁻ cell population was separated from the CD19 positive cell population; RBD positive memory B cells were separated from the mIg-G⁺mIg-D⁻ cell population, and sorted by a flow cytometric sorter.

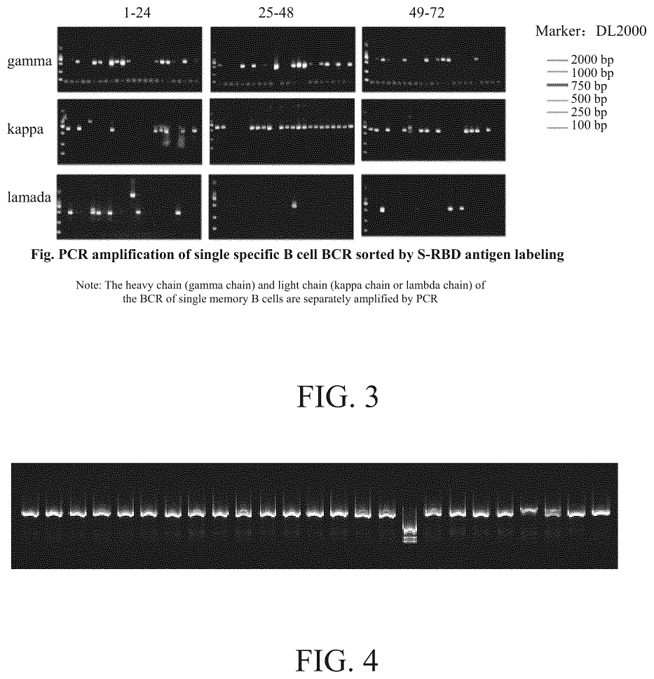

S3, the mRNA of a single RBD-specific memory B cell was collected, and the antibody variable region cDNA was obtained by RT-PCR amplification. Specifically, when the antibody variable region cDNA was subjected to the RT-PCR amplification, primers designed in this example efficiently facilitate amplification of an antibody gene sequence. Results are shown in FIG. 3.

S4, the antibody variable region cDNA obtained in S1-S3 was amplified by nested PCR to construct an antibody variable region gene expression cassette.

S3 and S4 were performed by following eight procedures: (1) freezing/thawing and lysis of cells; (2) preparation of related primers; (3) single-cell mRNA reverse transcription (RT); (4) a 1st PCR; (5) a 2nd PCR; (6) BCR-ORF PCR amplification for constructing a gene expression cassette; (7) CMV and WPRE-γ/κ/1 fragment amplification and CMV, BCR-Vγ/κ/1 (a product of (6)) and WPRE-γ/κ/1 overlapping PCR pre-ligation; and (8) BCR-γ ORF, BCR-κ ORF and BCR-1 PCR amplification.

Operations, preparation of reaction solutions and reaction conditions are as follows:
(1) After flow cytometric sorting, the cells were stored in a freezer at −80° C.; before RT-PCR, the cells were thawed to a room temperature, centrifuged at 300 g for 5 min, and kept in an ice bath for later use.
(2) Preparation of related primers (for primer sequences, see Table 9. Primer sequence listing)

Preparation of BCR_RT_Primer_Mix (each 2 μM): G289_primer (10 μM), K244_primer (10 μM) and L81 primer (10 μM), each of 100 μL, were taken and mixed in a ratio of 1:1:1.

Preparation of AP_Leader_Mix (each 2 μM): AP_G_Leader_Mix: 380 μL of water was added into a 1.5-mL centrifuge tube, 31 GV_N primers (each of 20 μL) were taken, with a final volume of 1000 μL; AP_K_Leader_Mix: 620 μL of water was added into a 1.5-mL centrifuge tube, 19 KV_N primers (each of 20 μL) were taken, with a final volume of 1000 μL; AP_L_Leader_Mix: 580 μL of water was added into a 1.5-mL centrifuge tube, 21 LV_N primers (each of 20 μL) were taken, with a final volume of 1000 μL;

Preparation of IGHJ_region_Primer_Mix: 920 μL of water was added into a 1.5-mL centrifuge tube, 4 IGHJ_N primers (each of 20 μL) were taken, with a final volume of 1000 μL;

Preparation of K194_Primer_Mix: 2 primers (K194-primer-01 (10 μM) and K194-primer-02 (10 μM)) were taken, each of 100 μL, and mixed;

Preparation of L19_Primer_Mix: 3 primers (L19-primer-01 (10 μM), L19-primer-02 (10 μM) and L19-primer-03 (10 μM)) were taken, each of 100 μL, and mixed.
(3) Reverse transcription (RT) (a 10-μL system): reagents required for the preparation are shown in Table 1 below.

TABLE 1
Experimental system of reverse transcription

| Seq. | Component | Amount (μl) |
|---|---|---|
| 1 | Water | 4.85 |
| 2 | 2.5 mM dNTPs | 2 |
| 3 | BCR_RT_Primer_Mix | 0.4 |
| 4 | 5 × PrimeScript II Buffer | 2 |
| 5 | 200 U/μl PrimeScript II Reverse Transcriptase | 0.5 |
| 6 | 40,000 U/ml RNase Inhibitor, Murine (NEB) | 0.25 |

10 μL of the reaction solution was added to a PCR tube containing cells, mixed well, and centrifuged at 300 g for 1 min.

Reaction conditions: at 45° C. for 45 min (mixing every 20 min); at 70° C. for 15 min.

After the reaction was complete, the 96-well plate was subjected to transient centrifugation at 600×g, and 1 μL of RT product was taken as a template for the 1st PCR.
(4) 1st PCR (a 10-μL system) (see a primer sequence listing for primer sequences): as shown in Table 2 below, 3 PCR systems were prepared for separate amplification of three antibody chains, respectively. The reagents and amounts added were the same except for the primers added.

TABLE 2
Experimental system of the 1st PCR

| Seq. | Component | Amount (μl) |
|---|---|---|
| 1 | 2 × PrimeSTAR GC Buffer (Takara) | 5 |
| 2 | nuclease-free water | 2.75 |
| 3 | 2.5 mM dNTP | 0.8 |
| 4 | 10 μM Forward Primer | 0.2 |
| 5 | 10 μM Reverse Primer | 0.2 |
| 6 | 2.5 U/μl PrimeSTAR HS DNA polymerase | 0.05 |
| 7 | Template (RT product solution) | 1 |

Primers to be added are shown in Table 3:

TABLE 3
Primers of the 1st PCR

| | Antibody | Antibody | Antibody |
|---|---|---|---|
| | gamma chain | kappa chain | lambda chain |
| | amplification | amplification | amplification |
| Forward | AP_G_leader | AP_K_leader | AP_L_leader |
| Primer | Mix | Mix | Mix |
| Reverse | G289_primer(10 | K244_Primer(10 | L81_Primer(10 |
| Primer | μM) | μM) | μM) |

Based on principle of PCR, experimental reaction conditions of the 1st PCR were: (1) pre-denaturation at 95° C. for 3 min; (2) denaturation at 95° C. for 10 s, annealing at 55° C. for 5 s, and extension at 72° C. for 1 min and for 30 cycles; and (3) extension for 5 min at 72° C. after the cycles, and storage at 4° C.
(5) 2nd PCR (a 10-μL system) (see a primer sequence listing, Tables 8 and 9, for primer sequences): as shown in Table 4 below, 3 PCR systems were prepared for separate amplification of three antibody chains, respectively. The reagents and amounts added were the same except for the primers added.

TABLE 4
Experimental system of the 2nd PCR

| Seq. | Component | Amount (μl) |
|---|---|---|
| 1 | 2 × PrimeSTAR GC Buffer (Takara) | 5 |
| 2 | nuclease-free water | 2.75 |
| 3 | 2.5 mM dNTP | 0.8 |
| 4 | 10 μM Forward Primer | 0.2 |
| 5 | 10 μM Reverse Primer | 0.2 |
| 6 | 2.5 U/μl PrimeSTAR HS DNA polymerase | 0.05 |
| 7 | Template (10-fold diluted product of the 1st PCR) | 1 |

Primers to be added are shown in Table 5:

TABLE 5
Primers of the 2nd PCR

| | | Antibody | Antibody |
|---|---|---|---|
| | Antibody gamma chain | kappa chain | lambda chain |
| | amplification | amplification | amplification |
| Forward | 10 μM AP_Primer | 10 μM | 10 μM |
| Primer | | AP_Primer | AP_Primer |
| Reverse | IGHJ_region_Primer | 10 μM | 10 μM |
| Primer | Mix | K194_Primer | L19_Primer |
| | | Mix | Mix |

Based on principle of PCR, experimental reaction conditions of the 2nd PCR were: (1) pre-denaturation at 95° C. for 3 min; (2) denaturation at 95° C. for 10 s, annealing at 55° C. for 5 s, and extension at 72° C. for 45 s and for 35 cycles; and (3) extension for 5 min at 72° C. after the cycles, and storage at 4° C.

After the PCR was finished, 4 μL of each well was loaded for 1.5% agarose gel electrophoresis. Wells of the paired Gamma, Kappa or Lambda chains were sequenced.
(6) Amplification and construction of an antibody expression cassette (BCR-ORF): for PCR amplification of a promoter region (CMV promoter), WPRE-γ (antibody gamma chain) and WPRE-κ (antibody kappa chain), PCR amplification systems are shown in Table 6 below.

TABLE 6
Amplification system

| CMV promoter PCR | | | WPRE-γ PCR | | | WPRE-κ/l PCR | | |
|---|---|---|---|---|---|---|---|---|
| System | | | system | | | system | | |
| 2xMix | 25 | μl | 2xMix | 25 | μl | 2xMix | 25 | μl |
| H₂O | 22 | μl | H₂O | 22 | μl | H₂O | 22 | μl |
| CMV-FP01 | 1 | μl | CMV-FP01 | 1 | μl | CMV-FP01 | 1 | μl |
| CMV-RP | 1 | μl | CMV-RP | 1 | μl | CMV-RP | 1 | μl |
| pcDNA3.4- | 1 μl | | pcDNA3.4- | 1 μl | | pcDNA3.4- | 1 μl | |
| ScaB-IgG1 | (25 ng) | | ScaB-IgG1 | (25 ng) | | ScaB-Hk/HL | (25 ng) | |
| Total | 50 | μl | Total | 50 | μl | Total | 50 | μl |

PCR amplification conditions were: (1) pre-denaturation at 95° C. for 3 min; (2) denaturation at 95° C. for 15 s, annealing at 56° C. for 15 s, and extension at 72° C. for 1 min and for 30 cycles; and (3) extension for 5 min at 72° C. after the cycles, and storage at 12° C.
(7) CMV and WPRE-γ/κ/1 fragment amplification and CMV, BCR-Vγ/κ/1 and WPRE-γ/κ/1 overlap PCR pre-ligation: experimental systems are shown in Table 7 below.

TABLE 7
Experimental system of amplification

| | |
|---|---|
| BCR-γ PCR system | BCR-κ/l PCR system |

| | | | | | |
|---|---|---|---|---|---|
| 2xMix | 12.5 | μl | 2xMix | 12.5 | μl |
| H₂O | 9 | μl | H₂O | 9 | μl |
| CMV | 1 | μl | CMV | 1 | μl |
| WPRE-γ | 1 | μl | WPRE-κ/l | 1 | μl |
| BCR-Vγ | 1.5 | μl | BCR-Vκ/l | 1.5 | μl |
| Total | 25 | μl | Total | 25 | μl |

PCR amplification conditions were: pre-denaturation at 95° C. for 3 min; denaturation at 95° C. for 15 s, annealing at 50° C. for 15 s, and extension at 72° C. for 1.5 min and for 10 cycles; and extension for 5 min at 72° C. after the cycles, and storage at 12° C.

(8) BCR-γ ORF, BCR-κ ORF and BCR-1 PCR amplification: experimental systems are shown in Table 8 below.

TABLE 8
Experimental system of amplification

| BCR-γ PCR system | | | BCR-κ/l PCR system | | |
|---|---|---|---|---|---|
| 2xMix | 25 | μl | 2xMix | 25 | μl |
| H₂O | 22.5 | μl | H₂O | 22.5 | μl |
| CMV-FP01 | 1 | μl | CMV-FP01 | 1 | μl |
| WPRE-RP | 1 | μl | WPRE-RP | 1 | μl |
| BCR-γ pre-ligation | 0.5 | μl | BCR-κ/l pre-ligation | 0.5 | μl |
| product | | | product | | |
| Total | 50 | μl | Total | 50 | μl |

PCR amplification procedures: pre-denaturation at 95° C. for 3 min; denaturation at 95° C. for 15 s, annealing at 58° C. for 15 s, and extension at 72° C. for 1.5 min, and for 30 cycles; and extension for 5 min at 72° C. after the cycles, and storage at 12° C.

After amplification, by agarose gel electrophoresis, whether the size of the obtained antibody variable region gene was correct or not was analyzed by gel imaging. The results are shown in FIG. 4, where a Marker is in a middle and a strip is at 5000 bp.

BCR-γ ORF and BCR-κ/ORF ethanol precipitation: PCR products of BCR-γ ORF and BCR-κ ORF (each of 30 μL) were added into an 8-tube strip before 120 μL of absolute ethanol and 6 μL of sodium acetate solution were added, which was uniformly stirred, standing for 30 min at −80° C. and centrifuged at 10000 rpm for 20 min. Supernatant was discarded, and the residue was sequentially washed with 200 μL of 70% ethanol and absolute ethanol once. Ethanol was evaporated at 56° C. 40 μL of sterile water was added and the mixture was shaken completely to dissolve the precipitate before the concentration of the antibody variable region gene was determined.

See Table 9 for the primer sequences.

TABLE 9
Primer sequence listing

| Primer_ | V/D/J gene | | |
|---|---|---|---|
| Name | segments | Primer_Sequence(5'>3') | Usage description |
| GV_01 | IGHV1-18*01 | CGGTACCGCGGGCCCGGGAatggactggacctggagcat | AP_G_Leader_Mix |
| | | (SEQ ID NO: 4) | As forward primer |
| | | | of heavy chain 1st |
| | | | PCR |
| GV_02 | IGHV1-2*01 | CGGTACCGCGGGCCCGGGAatggactggacctggaggat | |
| | | (SEQ ID NO: 5) | |
| GV_03 | IGHV1-24*01 | CGGTACCGCGGGCCCGGGAatggactgcacctggaggat | |
| | | (SEQ ID NO: 6) | |
| GV_04 | IGHV1-38-4*01 | CGGTACCGCGGGCCCGGGAatggactggaactggaggat | |
| | | (SEQ ID NO: 7) | |
| GV_05 | IGHV1-45*01 | CGGTACCGCGGGCCCGGGAatggactggacctggagaat | |
| | | (SEQ ID NO: 8) | |
| GV_06 | IGHV1-46*01 | CGGTACCGCGGGCCCGGGAatggactggacctggagggt | |
| | | (SEQ ID NO: 9) | |
| GV_07 | IGHV1-58*01 | CGGTACCGCGGGCCCGGGAatggactggatttggaggat | |
| | | (SEQ ID NO: 10) | |
| GV_08 | IGHV1-69*01 | CGGTACCGCGGGCCCGGGAatggactggacctggaggtt | |
| | | (SEQ ID NO: 11) | |
| GV_09 | IGHV2-26*01 | CGGTACCGCGGGCCCGGGAatggacacactttgctacac | |
| | | (SEQ ID NO: 12) | |
| GV_10 | IGHV2-5*01 | CGGTACCGCGGGCCCGGGAatggacacactttgctccac | |
| | | (SEQ ID NO: 13) | |
| GV_11 | IGHV2-70*01 | CGGTACCGCGGGCCCGGGAatggacatactttgttccac | |
| | | (SEQ ID NO: 14) | |
| GV_12 | IGHV2/OR16- | CGGTACCGCGGGCCCGGGAatggacacgttttgctccac | |
| | 5*01 | (SEQ ID NO: 15) | |
| GV_13 | IGHV3-11*01 | CGGTACCGCGGGCCCGGGAatggagtttgggctgagctg | |
| | | (SEQ ID NO: 16) | |
| GV_14 | IGHV3-13*01 | CGGTACCGCGGGCCCGGGAatggagttggggctgagctg | |
| | | (SEQ ID NO: 17) | |
| GV_15 | IGHV3-16*01 | CGGTACCGCGGGCCCGGGAatggaatttgggctgagctg | |
| | | (SEQ ID NO: 18) | |
| GV_16 | IGHV3-21*01 | CGGTACCGCGGGCCCGGGAatggaactggggctccgctg | |
| | | (SEQ ID NO: 19) | |
| GV_17 | IGHV3-43*01 | CGGTACCGCGGGCCCGGGAatggagtttggactgagctg | |
| | | (SEQ ID NO: 20) | |
| GV_18 | IGHV3-48*01 | CGGTACCGCGGGCCCGGGAatggagttggggctgtgctg | |
| | | (SEQ ID NO: 21) | |
| GV_19 | IGHV3-49*01 | CGGTACCGCGGGCCCGGGAatggagtttgggcttagctg | |
| | | (SEQ ID NO: 22) | |
| GV_20 | IGHV3-53*01 | CGGTACCGCGGGCCCGGGAatggagttttggctgagctg | |
| | | (SEQ ID NO: 23) | |
| GV_21 | IGHV3-64*01 | CGGTACCGCGGGCCCGGGAatgacggagtttgggctgag | |
| | | (SEQ ID NO: 24) | |
| GV_22 | IGHV3-64D*06 | CGGTACCGCGGGCCCGGGAatggagttctggctgagctg | |
| | | (SEQ ID NO: 25) | |
| GV_23 | IGHV3-7*01 | CGGTACCGCGGGCCCGGGAatggaattggggctgagctg | |
| | | (SEQ ID NO: 26) | |
| GV_24 | IGHV3-9*01 | CGGTACCGCGGGCCCGGGAatggagttgggactgagctg | |
| | | (SEQ ID NO: 27) | |
| GV_25 | IGHV4-28*01 | CGGTACCGCGGGCCCGGGAatgaaacacctgtggttctt | |
| | | (SEQ ID NO: 28) | |
| GV_26 | IGHV4-38-2*02 | CGGTACCGCGGGCCCGGGAatgaagcacctgtggttttt | |
| | | (SEQ ID NO: 29) | |
| GV_27 | IGHV4-39*01 | CGGTACCGCGGGCCCGGGAatgaagcacctgtggttctt | |
| | | (SEQ ID NO: 30) | |
| GV_28 | IGHV4-59*01 | CGGTACCGCGGGCCCGGGAatgaaacatctgtggttctt | |
| | | (SEQ ID NO: 31) | |
| GV_29 | IGHV5-10-1*02 | CGGTACCGCGGGCCCGGGAatgcaagtgggggcctctcc | |
| | | (SEQ ID NO: 32) | |
| GV_30 | IGHV5-51*01 | CGGTACCGCGGGCCCGGGAatggggtcaaccgccatcct | |
| | | (SEQ ID NO: 33) | |
| GV_31 | IGHV6-1*01 | CGGTACCGCGGGCCCGGGAatgtctgtctccttcctcat | |
| | | (SEQ ID NO: 34) | |
| KV_01 | IGKV1/OR2- | CGGTACCGCGGGCCCGGGAatgagggcccccactcagct | AP_K_Leader_Mix |
| | 0*01 | (SEQ ID NO: 35) | As forward primer |
| | | | of kappa light |
| | | | chain 1st PCR |
| KV_02 | IGKV1/OR2- | CGGTACCGCGGGCCCGGGAatggaaatgagggtccccgc | |
| | 108*01 | (SEQ ID NO: 36) | |
| KV_03 | IGKV1-16*01 | CGGTACCGCGGGCCCGGGAatggacatgagagtcctcgc | |
| | | (SEQ ID NO: 37) | |
| KV_04 | IGKV1-27*01 | CGGTACCGCGGGCCCGGGAatggacatgagggtccctgc | |
| | | (SEQ ID NO: 38) | |
| KV_05 | IGKV1-5*01 | CGGTACCGCGGGCCCGGGAatggacatgagggtccccgc | |
| | | (SEQ ID NO: 39) | |
| KV_06 | IGKV1-8*01 | CGGTACCGCGGGCCCGGGAatgagggtccccgctcagct | |
| | | (SEQ ID NO: 40) | |
| KV_07 | IGKV1D-16*01 | CGGTACCGCGGGCCCGGGAatggacatgagggtcctcgc | |
| | | (SEQ ID NO: 41) | |
| KV_08 | IGKV1D-43*01 | CGGTACCGCGGGCCCGGGAatggacatgagggtgcccgc | |
| | | (SEQ ID NO: 42) | |
| KV_09 | IGKV2-24*01 | CGGTACCGCGGGCCCGGGAatgaggctccttgctcagct | |
| | | (SEQ ID NO: 43) | |
| KV_10 | IGKV2-28*01 | CGGTACCGCGGGCCCGGGAatgaggctccctgctcagct | |
| | | (SEQ ID NO: 44) | |
| KV_11 | IGKV3/OR2- | CGGTACCGCGGGCCCGGGAatggaagccccagcacagct | |
| | 268*01 | (SEQ ID NO: 45) | |
| KV_12 | IGKV3-15*01 | CGGTACCGCGGGCCCGGGAatggaagccccagcgcagct | |
| | | (SEQ ID NO: 46) | |
| KV_13 | IGKV3-20*01 | CGGTACCGCGGGCCCGGGAatggaaaccccagcgcagct | |
| | | (SEQ ID NO: 47) | |
| KV_14 | IGKV3-7*01 | CGGTACCGCGGGCCCGGGAatggaagccccagctcagct | |
| | | (SEQ ID NO: 48) | |
| KV_15 | IGKV3D-7*01 | CGGTACCGCGGGCCCGGGAatggaaccatggaagcccca | |
| | | (SEQ ID NO: 49) | |
| KV_16 | IGKV4-1*01 | CGGTACCGCGGGCCCGGGAatggtgttgcagacccaggt | |
| | | (SEQ ID NO: 50) | |
| KV_17 | IGKV5-2*01 | CGGTACCGCGGGCCCGGGAatggggtcccaggttcacct | |
| | | (SEQ ID NO: 51) | |
| KV_18 | IGKV6-21*01 | CGGTACCGCGGGCCCGGGAatgttgccatcacaactcat | |
| | | (SEQ ID NO: 52) | |
| KV_19 | IGKV6D-41*01 | CGGTACCGCGGGCCCGGGAatggtgtccccgttgcaatt | |
| | | (SEQ ID NO: 53) | |
| LV_01 | IGLV1-40*01 | CGGTACCGCGGGCCCGGGAatggcctggtctcctctcct | AP_L_Leader_Mix |
| | | (SEQ ID NO: 54) | As forward primer |
| | | | of lambda light |
| | | | chain 1st PCR |
| LV_02 | IGLV1-41*01 | CGGTACCGCGGGCCCGGGAatgacctgctcccctetcct | |
| | | (SEQ ID NO: 55) | |
| LV_03 | IGLV1-47*02 | CGGTACCGCGGGCCCGGGAatggccggcttccctctcct | |
| | | (SEQ ID NO: 56) | |
| LV_04 | IGLV10-54*02 | CGGTACCGCGGGCCCGGGAatgccctgggctctgctcct | |
| | | (SEQ ID NO: 57) | |
| LV_05 | IGLV11-55*01 | CGGTACCGCGGGCCCGGGAatggccctgactcctctcct | |
| | | (SEQ ID NO: 58) | |
| LV_06 | IGLV2-8*02 | CGGTACCGCGGGCCCGGGAatggcctgggctctgctgct | |
| | | (SEQ ID NO: 59) | |
| LV_07 | IGLV3-1*01 | CGGTACCGCGGGCCCGGGAatggcatggatccctctctt | |
| | | (SEQ ID NO: 60) | |
| LV_08 | IGLV3-10*02 | CGGTACCGCGGGCCCGGGAatggcctggacccctctcct | |
| | | (SEQ ID NO: 61) | |
| LV_09 | IGLV3-19*01 | CGGTACCGCGGGCCCGGGAatggcctggacccctctctg | |
| | | (SEQ ID NO: 62) | |
| LV_10 | IGLV3-21*01 | CGGTACCGCGGGCCCGGGAatggcctggaccgttctcct | |
| | | (SEQ ID NO: 63) | |
| LV_11 | IGLV3-25*02 | CGGTACCGCGGGCCCGGGAatggcctggatccctctact | |
| | | (SEQ ID NO: 64) | |
| LV_12 | IGLV3-27*01 | CGGTACCGCGGGCCCGGGAatggcctggatccctctcct | |
| | | (SEQ ID NO: 65) | |
| LV_13 | IGLV3-9*02 | CGGTACCGCGGGCCCGGGAatggcctggaccgctctcct | |
| | | (SEQ ID NO: 66) | |
| LV_14 | IGLV4-3*01 | CGGTACCGCGGGCCCGGGAatggcctgggtctccttcta | |
| | | (SEQ ID NO: 67) | |
| LV_15 | IGLV4-60*02 | CGGTACCGCGGGCCCGGGAatggcctggaccccactcct | |
| | | (SEQ ID NO: 68) | |
| LV_16 | IGLV5-39*02 | CGGTACCGCGGGCCCGGGAatggcctggactcctctcct | |
| | | (SEQ ID NO: 69) | |
| LV_17 | IGLV6-57*02 | CGGTACCGCGGGCCCGGGAatggcctgggctccactact | |
| | | (SEQ ID NO: 70) | |
| LV_18 | IGLV7-43*01 | CGGTACCGCGGGCCCGGGAatggcctggactcctctctt | |
| | | (SEQ ID NO: 71) | |
| LV_19 | IGLV8-61*02 | CGGTACCGCGGGCCCGGGAatggcctggatgatgcttct | |
| | | (SEQ ID NO: 72) | |
| LV_20 | IGLV8/OR8- | CGGTACCGCGGGCCCGGGAatggcctgcatgatgcttct | |
| | 1*02 | (SEQ ID NO: 73) | |
| LV_21 | IGLV9-49*02 | CGGTACCGCGGGCCCGGGAatggcctgggctcctctgct | |
| | | (SEQ ID NO: 74) | |
| IGHJ_01 | IGHJ1*01 | GATGGGCCCTTGGTGGAGGGTGAGGAGACGGTGA | IGHJ_region_ |
| | | CCAGGG(SEQ ID NO: 75) | Primer_Mix |
| IGHJ_02 | IGHJ2*01 | GATGGGCCCTTGGTGGAGGGTGAGGAGACAGTGA | |
| | | CCAGGG(SEQ ID NO: 76) | |
| IGHJ_03 | IGHJ3*01 | GATGGGCCCTTGGTGGAGGGTGAAGAGACGGTGA | |
| | | CCATTG(SEQ ID NO: 77) | |
| IGHJ_04 | IGHJ6*01 | GATGGGCCCTTGGTGGAGGGTGAGGAGACGGTGA | |
| | | CCGTGG(SEQ ID NO: 78) | |
| IGKJ_01 | IGKJ1*01 | GATGGTGCAGCCACAGTTCGTTTGATTTCCACCTTG | IGKJ_region_ |
| | | GTCC(SEQ ID NO: 79) | Primer_Mix |
| IGKJ_02 | IGKJ2*01 | GATGGTGCAGCCACAGTTCGTTTGATCTCCAGCTTG | |
| | | GTCC(SEQ ID NO: 80) | |
| IGKJ_03 | IGKJ3*01 | GATGGTGCAGCCACAGTTCGTTTGATATCCACTTTG | |
| | | GTCC(SEQ ID NO: 81) | |
| IGKJ_04 | IGKJ4*01 | GATGGTGCAGCCACAGTTCGTTTGATCTCCACCTTG | |
| | | GTCC(SEQ ID NO: 82) | |
| IGKJ_05 | IGKJ5*01 | GATGGTGCAGCCACAGTTCGTTTAATCTCCAGTCGT | |
| | | GTCC(SEQ ID NO: 83) | |
| IGLJ_01 | IGLJ1*01 | GGGGCAGCCTTGGGCTGACCTAGGACGGTGACCTT | IGLJ_region_ |
| | | GGTCC(SEQ ID NO: 84) | Primer_Mix |
| IGLJ_02 | IGLJ2*01 | GGGGCAGCCTTGGGCTGACCTAGGACGGTCAGCTT | |
| | | GGTCC(SEQ ID NO: 85) | |
| IGLJ_03 | IGLJ4*01 | GGGGCAGCCTTGGGCTGACCTAAAATGATCAGCTG | |
| | | GGTTC(SEQ ID NO: 86) | |
| IGLJ_04 | IGLJ5*01 | GGGGCAGCCTTGGGCTGACCTAGGACGGTCAGCTC | |
| | | GGTCC(SEQ ID NO: 87) | |
| IGLJ_05 | IGLJ5*02 | GGGGCAGCCTTGGGCTGACCTAGGACGGTCAGCTC | |
| | | CGTCC(SEQ ID NO: 88) | |
| IGLJ_06 | IGLJ6*01 | GGGGCAGCCTTGGGCTGACCGAGGACGGTCACCTT | |
| | | GGTGC(SEQ ID NO: 89) | |
| IGLJ_07 | IGLJ7*01 | GGGGCAGCCTTGGGCTGACCGAGGACGGTCAGCT | |
| | | GGGTGC(SEQ ID NO: 90) | |
| IGLJ_08 | IGLJ7*02 | GGGGCAGCCTTGGGCTGACCGAGGGCGGTCAGCT | |
| | | GGGTGC(SEQ ID NO: 91) | |
| G289-primer | N.D. | TCTTGTCCACCTTGGTGTTGCT | As reverse primer |
| | | (SEQ ID NO: 92) | of heavy chain RT |
| | | | & 1st PCR |
| G97-primer | N.D. | AGTAGTCCTTGACCAGGCAGCCCAG | As reverse primer |
| | | (SEQ ID NO: 93) | of heavy chain 2nd |
| | | | PCR |
| K244-primer | N.D. | GTTTCTCGTAGTCTGCTTTGCTCA | As reverse primer |
| | | (SEQ ID NO: 94) | of kappa light chain |
| | | | RT & 1st PCR |
| K194-primer- | N.D. | GTGCTGTCCTTGCTGTCCTGCT | As reverse primer |
| 01 | | (SEQ ID NO: 95) | of kappa light chain |
| | | | 2nd PCR |
| K194-primer- | N.D. | GTGCTGTCCTTGCTCTCCTGCT | |
| 02 | | (SEQ ID NO: 96) | |
| L81-primer | N.D. | CACCAGTGTGGCCTTGTTGGCTTG | As reverse primer |
| | | (SEQ ID NO: 97) | of lambda light |
| | | | chain RT & 1st PCR |
| L19-primer- | N.D. | GGGCGGGAACAGAGTGACC | As reverse primer |
| 01 | | (SEQ ID NO: 98) | of lambda light |
| | | | chain 2nd PCR |
| L19-primer- | N.D. | GGGCGGGAACAGAGTGACA | |
| 02 | | (SEQ ID NO: 99) | |
| L19-primer- | N.D. | GGGTGGGAACAGAGTGACC | |
| 03 | | (SEQ ID NO: 100) | |
| AP3 | N.D. | CGGTACCGCGGGCCCGGGA | As forward primer |
| | | (SEQ ID NO: 101) | of 2nd PCR |
| G20FP | N.D. | CCCTCCACCAAGGGCCCATC | Construction of the |
| | | (SEQ ID NO: 102) | linear antibody |
| | | | expression cassettes |

S5, the antibody variable region gene expression cassette obtained in S4 was transfected into 293T cells for antibody expression in 48 h; supernatant was collected, and the RBD specificity was determined by ELISA to select full human monoclonal antibodies of RBD specificity.

(A) The antigen was diluted with PBS to a final concentration of 2 μg/mL, and a 384-well ELISA plate was coated at 10 μL/well overnight at 4° C. or for 2 h at 37° C. (overnight at 4° C. is preferred in this example). NOTE: After the addition, the plate was subjected to transient centrifugation to ensure that the liquid was at bottom.

An experimental system is shown in Table 10 below:

TABLE 10
Experimental system of coating

| | | Original | | Final | Dilution |
|---|---|---|---|---|---|
| Reagent | Catalog No. | concentration | | concentration | factor |
| SARS-COV-2 | Cat: 40592- | 200 | μg/mL | 2 μg/mL | 1:100 |
| RBD | V08H | | | | |
| Goat pab to | Cat: ab97221 | 1 | mg/mL | 2 μg/mL | 1:500 |
| Hu IgG-ALP | | | | | |

(B) Preparation of PBST (0.05% of Tween 20, Cat. No. TB220): 0.5 mL of Tween 20 was added for 1 L of PBS;

The plate was washed with PBST in a Thermo Scientific Wellwash Versa microplate washer or manually (the machine-washed plate was also manually clapped/centrifuged for 1 min on a microplate centrifuge (MPC-P25) until no water or bubbles were found on the plate).

Blocking: 80 μL of 5% BSA (BioFroxx, Cat. No. 4240GR100) in PBST were added to the washed plate and incubated for 1 h at 37° C. in an incubator. The plate was washed with PBST by machine or manually.

(C) Adding samples and standards. Standard: 10 μL/well, serially diluted from an original concentration of 1 μg/mL to 250 ng/mL, 125 ng/mL, 62.5 ng/mL, 31.25 ng/mL, 15.63 ng/mL, 7.81 ng/mL, 3.9 ng/mL and 1.95 ng/mL. Blocking solution; Sample: supernatant of cells transfected with antibody gene. Negative control/blank control: blocking solution, at 10 μL/well.

The plate was incubated at 37° C. for 30 min, and washed with PBST by machine or manually.

(D) Adding secondary antibody: at a concentration of 10 μL/well, followed by incubation at 37° C. for 30 min. An experimental system is shown in Table 11 below:

TABLE 11
Experimental system of secondary antibody

| Name of | | | | |
|---|---|---|---|---|
| Secondary | Catalog | Original | Final | Dilution |
| antibody | No. | concentration | concentration | factor |
| goat-anti-human | A18808 | 1.5 mg/ml | 0.3 μg/ml | 1:5000 |
| IgG-ALP | | | | |
| Goat pab to Hu | Ab98532 | 0.5 mg/ml | 0.25 μg/ml | 1:2000 |
| IgG-ALP | | | | |

The plate was washed with PBST by machine or manually. PNPP (disodium p-nitrophenyl phosphate) was added at 10 μL/well and the OD (450 nm) values were measured using a Thermo Scientific Muttiskan GO system at 5 min, 10 min, 15 min, 20 min, 25 min, 30 min, 35 min, 40 min, 45 min, 50 min, 55 min and 60 min. 50 mg of PNPP powder (Thermo, Prod. No. 34045) and 40 mL of ddH2O and 10 mL of diethanol amine substrate buffer (5x). PNPP was stored away from light at 4° C.

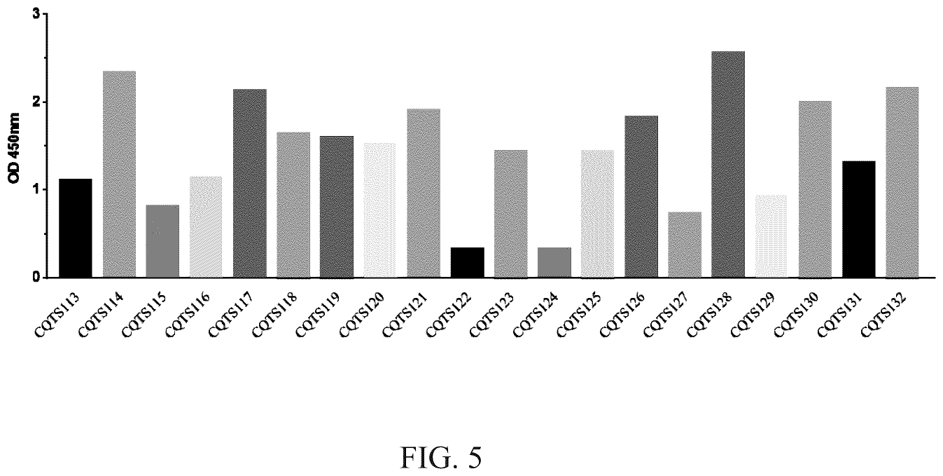

As shown in FIG. 5, CQTS126 is a desired monoclonal antibody. Samples with an OD value of 0.1 or higher are positive.

### Example 2

This example provides a linear epitope of the SARS-CoV-2 RBD-specific monoclonal antibody having an amino acid sequence set forth in SEQ ID NO: 3.

This example further provides use of the linear epitope of the SARS-CoV-2 RBD-specific monoclonal antibody in preparing a nucleic acid, a recombinant vector, a host cell, a composition, a vaccine, test paper, a test reagent or a monoclonal antibody.

This example further provides a method for screening the linear epitope of the SARS-CoV-2 RBD-specific monoclonal antibody. At present, many documents have reported that the key amino acids or amino acid sites for the spatial structure of the SARS-CoV-2 S protein, RBD protein, and antibodies and receptor ACE2 of the proteins can be analyzed through the structure, but only a few are about the linear epitope of SARS-CoV-2. For example, “Mining of epitopes on spike protein of SARS-CoV-2 from COVID-19 patients” (Cell Research (2020) 0:1-3; https://doi org/10.1038/s41422-020-0366-x) reports the prediction of epitopes in S protein through softwares and the analysis using the blood of COVID-19 patients in convalescence, but not monoclonal antibody verification. In this example, a different method for designing epitopes was used, and a different linear epitope from known ones was found. In this experiment, SARS-CoV-2 S protein or SARS-CoV-2 RBD protein was denatured; the SARS-CoV-2 RBD-specific monoclonal antibody was bound to the S protein or the RBD protein after denaturation; an antigenic linear epitope section of the S protein or the RBD protein was then synthesized to give the linear epitope. The experiment specifically comprises the following steps:
1. Linear epitopes were designed and synthesized;
2. Antibody binding capacity of the linear epitopes was detected by an ELISA method to screen the linear epitopes. Specific principle is as follows: if the epitope of the antibody is a steric epitope, stereo conformation may be destroyed by treatment with SDS, mercaptoethanol, DTT, etc., and cannot be identified by the antibody. If the epitope is a linear epitope, the antibody can still bind to the epitope.
(1) Consumables and reagents: streptavidin-coated plates (Thermo Fisher, P #15504); a Wash Buffer (prepared with Pierce® Protein-Free Blocking Buffers, P #37573), with 0.1% of BSA (BioFroxx, P #) added; goat F(ab′)2 anti-human IgG (Fab′)2 (alkaline phosphatase, Abcam, P #ab98532); and PNPP substrate (Thermo Fisher, P #34045).
(2) A first day: the chemically synthesized RBD antigen peptide was diluted with PBS (with a final concentration of 20 μg/mL), and a 384-well ELISA plate was coated at 10 μL/well overnight at 4° C. or for 2 h at 37° C. (overnight at 4° C. is preferred in this example). NOTE: After addition, the plate was subjected to transient centrifugation.

The coated plate was High Binding, CORNING, Lot No. 20519008.
(3) A second day:
(a1) Preparation of PBST (0.05% of Tween 20, Cat. No. TB220): 0.5 mL of Tween 20 was added for 1 L of PBS;

The plate was washed with PBST in a Thermo Scientific Wellwash Versa microplate washer or manually (the machine-washed plate was also manually clapped/centrifuged for 1 min on a microplate centrifuge (MPC-P 25) until no water or bubbles were found on the plate);
(a2) Blocking: the plate was blocked with 80 μL of 5% BSA (BioFroxx, Cat. No. 4240GR100) (PBST preparation) for 1 h at 37° C.;
(a3) SARS-CoV-2 RBD-specific monoclonal antibodies were added at 10 μL/well (20 μg/mL), and the plate was washed 5 times after 1 h at the room temperature;
(a4) 50 μL of goat F(ab′)2 anti-human IgG (Fab′)2 (alkaline phosphatase; Abcam, P #ab98532) was added, and the plate was incubated for 30 min at the room temperature;
(a5) The plate was washed with 100 μL of wash buffer 5 times, and 50 μL of reaction substrate PNPP was added;
(a6) An absorbance value (OD 405 nm) was measured.

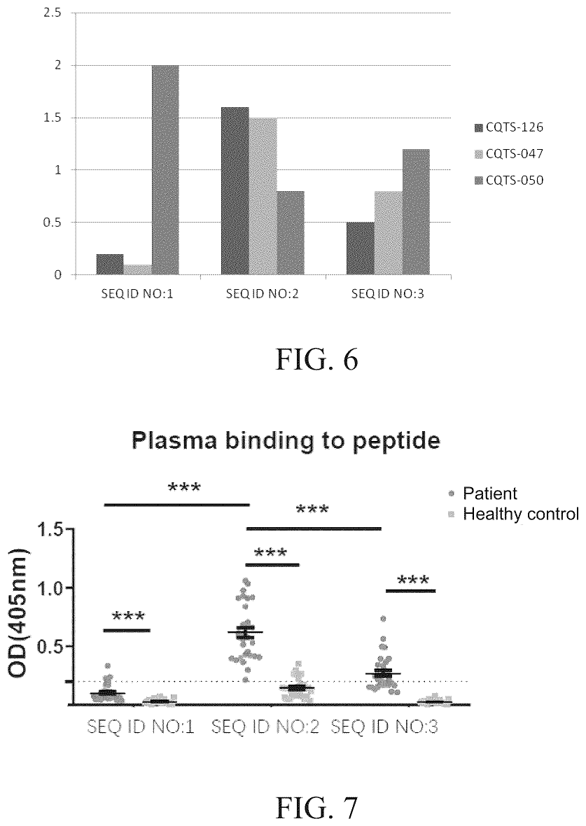

Conclusion: as shown in FIG. 6, linear epitopes of SEQ ID NO: 3 were screened by experimental results of whether the SARS-CoV-2 RBD-specific monoclonal antibodies CQTS047, CQTS050 and CQTS126 bound to the epitope or not.

Experiment I: Linear epitope detection using serum antibody from COVID-19 patients in convalescence
(1) Consumables and reagents: streptavidin-coated plates (Thermo Fisher, P #15504); a Wash Buffer (prepared with Pierce® Protein-Free Blocking Buffers, P #37573), with 0.1% of BSA (BioFroxx, P #) added; goat F(ab′)2 anti-human IgG (Fab′)2 (alkaline phosphatase, Abcam, P #ab98532); and PNPP substrate (Thermo Fisher, P #34045);
(2) A first day: the chemically synthesized RBD antigen peptide was diluted with PBS (with a final concentration of 20 μg/mL), and a 384-well ELISA plate was coated at 10 μL/well overnight at 4° C. or for 2 h at 37° C. (overnight at 4° C. is preferred in this example). NOTE: After the addition, the plate was subjected to transient centrifugation.

The coated plate was High Binding, CORNING, Lot No. 20519008.
(3) A second day:
(a1) Preparation of PBST (0.05% Tween 20, Cat. No. TB220): 0.5 mL of Tween 20 was added for 1 L of PBS;

The plate was washed with PBST in a Thermo Scientific Wellwash Versa microplate washer or manually (the machine-washed plate was also manually clapped/centrifuged for 1 min on a microplate centrifuge (MPC-P 25) until no water or bubbles were found on the plate);
(a2) Blocking: the plate was blocked with 80 μL of 5% BSA (BioFroxx, Cat. No. 4240GR100) (PBST preparation) for 1 h at 37° C.;
(a3) Serum of COVID-19 patients in convalescence and healthy subjects (negative control) were added at 10 μL/well (original concentration), and the plate was washed 5 times after 1 h of incubation at the room temperature;
(a4) 50 μL of goat F(ab′)2 anti-human IgG (Fab′)2 (alkaline phosphatase; Abcam, P #ab98532) was added, and the plate was incubated for 30 min at the room temperature;
(a5) The plate was washed with 100 μL of wash buffer 5 times, and 50 μL of reaction substrate PNPP was added;
(a6) An absorbance value (OD405 nm) was measured.

Conclusion: as shown in FIG. 7, the SARS-CoV-2 linear epitope of SEQ ID NO: 3 bound to serum of patients, but not to those of healthy subjects.

Experiment II: Binding of linear epitope to ACE2
(1) A first day: ACE2 protein (purchased from Sino Biological) was diluted with PBS (with a final concentration of 2 μg/mL), and a 384-well ELISA plate was coated at 10 μL/well overnight at 4° C. or for 2 h at 37° C. (overnight at 4° C. is preferred in this example). NOTE: After the addition, the plate was subjected to transient centrifugation.
(2) A second day:
(a1) Preparation of PBST (0.05% Tween 20, Cat. No. TB220): 0.5 mL of Tween 20 was added for 1 L of PBS;
The plate was washed with PBST in a Thermo Scientific Wellwash Versa microplate washer or manually (the machine-washed plate was also manually clapped/centrifuged for 1 min on a microplate centrifuge (MPC-P 25) until no water or bubbles were found on the plate);
(a2) Blocking: the plate was blocked with 80 μL of 5% BSA (BioFroxx, Cat. No. 4240GR100) (PBST preparation) for 1 h at 37° C.;
(a3) The RBD antigen peptide was added at 10 μL/well (20 μg/mL). The plate was washed 5 times after 1 h of incubation at the room temperature;
(a4) 50 μL of streptavidin-ALP antibody (3310-10) (1:1000) was added and the plate was incubated at the room temperature for 30 min;
(a5) The plate was washed with 100 μL of wash buffer 5 times, and 50 μL of reaction substrate PNPP was added;
(a6) An absorbance value (OD405 nm) was measured.

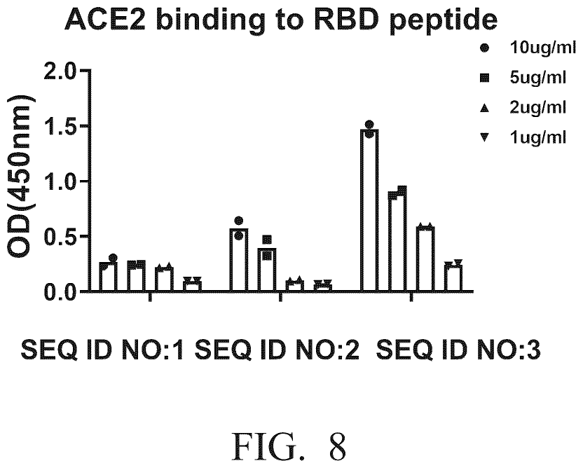

Conclusion: as shown in FIG. 8, the RBD antigen peptide of SEQ ID NO: 3 bound to ACRE2 of the RBD receptor.

Detailed above are preferred embodiments of the present disclosure. It should be appreciated that numerous modifications and variations can be devised by those skilled in the art according to the teachings of the present disclosure without creative efforts. Therefore, the technical schemes that can be obtained by those skilled in the art through logical analysis, inference or limited experiments based on the prior art according to the concepts of the present disclosure shall fall within the protection scope defined by the claims.

```
CQMU01-seql.txt
<110>CHONGQING MEDICAL UNIVERSITY, Yulin Feng
<120>NOVEL CORONAVIRUS RBD SPECIFIC MONOCLONAL
ANTIBODY AND LINEAR EPITOPE
AND APPLICATION THEREOF
<160>3
<210>1
<211>123
<212>PRT
<213>Artificial Sequence
<400>1
Gln Met Gln Leu Val Gln Ser Gly Pro Glu Val Lys Lys Pro Gly
1 5 10 15
Thr Ser Val Lys Val Ser Cys Lys Ala Ser Gly Phe Thr Phe Ser
                                20 25 30
Ser Ser Ala Val Gln Trp Val Arg Gln Ala Arg Gly Gln His Leu
                                35 40 45
Glu Trp Ile Gly Trp Ile Val Val Gly Ser Gly Asn Thr Asn Tyr
                                50 55 60
Ala Gln Lys Phe Gln Glu Arg Val Thr Leu Thr Arg Asp Met
                                65 70
Ser Thr Arg Thr Ala Tyr Met Glu Leu Ser Ser Leu Arg Ser Glu
75 80 85
Asp Thr Ala Val Tyr Tyr Cys Ala Ala Pro Asn Cys Asn Ser Thr
90 95 100
Thr Cys His Asp Gly Phe Asp Ile Trp Gly Gln Gly Thr Val Val
105 110 115
Thr Val Ser Ser
120
<210>2
<211>108
<212>PRT
<213>Artificial Sequence
<400>2
Glu Ile Val Leu Thr Gln Ser Pro Gly Thr Leu Ser Leu Ser Pro
1 5 10 15
Gly Glu Arg Ala Thr Leu Ser Cys Arg Ala Ser Gln Ser Val Arg
                                20 25 30
Ser Ser Tyr Leu Gly Trp Tyr Gln Gln Lys Pro Gly Gln Ala Pro
                                35 40 45
Arg Leu Leu Ile Tyr Gly Ala Ser Ser Arg Ala Thr Gly Ile Pro
                                50 55 60
Asp Arg Phe Ser Gly Ser Gly Ser Gly Thr Asp Phe Thr Leu Thr
                                65 70 75
Ile Ser Arg Leu Glu Ser Glu Asp Phe Ala Val Tyr Tyr Cys Gln
                                80 85 90
Gln Tyr Asp Asn Ser Pro Trp Thr Phe Gly Gln Gly Thr Lys Val
                                95 100 105
Glu Ile Lys
<210>3
<211>25
<212>PRT
<213>Artificial Sequence
<400>3
Ile Ala Trp Asn Ser Lys Val Gly Gly Asn Tyr Asn Tyr Leu Tyr
  1 5 10 15
Arg Leu Phe Arg Lys Ser Asn Leu Lys Pro
                                20 25
```

1. A SARS-CoV-2 RBD-specific monoclonal antibody, comprising a heavy chain having an amino acid sequence set forth in SEQ ID NO: 1 and a light chain having an amino acid sequence set forth in SEQ ID NO: 2.

2. The SARS-CoV-2 RBD-specific monoclonal antibody according to claim 1, wherein the SARS-CoV-2 RBD-specific monoclonal antibody is obtained by sorting RBD-specific memory B cells and then acquiring antibody variable region cDNA from mRNA of the RBD-specific memory B cells.

3. The SARS-CoV-2 RBD-specific monoclonal antibody according to claim 1, wherein the SARS-CoV-2 RBD-specific monoclonal antibody is for preparing a reagent or a medicament for detecting or diagnosing SARS-CoV-2.

4. (canceled)

5. (canceled)

6. The SARS-CoV-2 RBD-specific monoclonal antibody according to claim 1, wherein the SARS-CoV-2 RBD-specific monoclonal antibody is used for any of the following (b1)-(b4): (b1) binding to SARS-CoV-2; (b2) detecting SARS-CoV-2; (b3) binding to a SARS-CoV-2 S protein; and (b4) detecting the SARS-CoV-2 S protein.

7. The SARS-CoV-2 RBD-specific monoclonal antibody according to claim 1, wherein the SARS-CoV-2 RBD-specific monoclonal antibody comprises a linear epitope having an amino acid sequence set forth in SEQ ID NO: 3.

8. The SARS-CoV-2 RBD-specific monoclonal antibody according to claim 6, wherein the linear epitope is obtained by: denaturing SARS-CoV-2 S protein or SARS-CoV-2 RBD protein; binding the SARS-CoV-2 RBD-specific monoclonal antibody to the SARS-CoV-2 S protein or the SARS-CoV-2 RBD protein after denaturation; and performing antigenic linear epitope section synthesizing of the S protein or the RBD protein to obtain the linear epitope.

9. (canceled)

10. (canceled)

11. The SARS-CoV-2 RBD-specific monoclonal antibody according to claim 1, wherein the SARS-CoV-2 RBD-specific monoclonal antibody is for preparing a reagent or a medicament for detecting or diagnosing SARS-CoV-2.

12. The SARS-CoV-2 RBD-specific monoclonal antibody according to claim 11, wherein the medicament comprises the SARS-CoV-2 RBD-specific monoclonal antibody and a pharmaceutically acceptable excipient, diluent or carrier.

13. The SARS-CoV-2 RBD-specific monoclonal antibody according to claim 2, wherein the SARS-CoV-2 RBD-specific monoclonal antibody is for preparing a reagent or a medicament for detecting or diagnosing SARS-CoV-2.

14. The SARS-CoV-2 RBD-specific monoclonal antibody according to claim 13, wherein the medicament comprises the SARS-CoV-2 RBD-specific monoclonal antibody and a pharmaceutically acceptable excipient, diluent or carrier.
